(19) United States
(12) Patent Application Publication
(10) Pub. No.: US 2010/0023074 A1
(43) Pub. Date: Jan. 28, 2010

(54) Method and Apparatus for Contacting an Over-the-Counter Automatic External Defibrillator

(75) Inventors: Daniel J. Powers, Issaquah, WA (US); Teresa Skarr, Windsor, CA (US)

Correspondence Address: PHILIPS INTELLECTUAL PROPERTY & STANDARDS, P.O. BOX 3001, Briarcliff Manor, NY, 10510-8001, US

(73) Assignee: KONINKLIJKE PHILIPS ELECTRONICS N.V., EINDHOVEN (NL)

(21) Appl. No.: 11/722,212

(22) PCT Filed: Dec. 16, 2005
(86) PCT No.: PCT/IB2005/054292

§ 371 Date: Jun. 20, 2007

Related U.S. Application Data
(60) Provisional application No. 60/639,476, filed on Dec. 27, 2004.
(60) Provisional application No. 60/689,116, filed on Jun. 8, 2005.

(51) Int. Cl. A61N 1/39

(52) U.S. Cl. 607/5

(57) ABSTRACT

A method and apparatus are described by which messages can be sent wirelessly or over communication lines to an AED located in a home or office. The AED contains a receiver for receiving short messages directing the owner to call for service, conduct maintenance such as battery or electrode pad replacement, respond to an emergency or locate the AED. A wireless receiver enables the AED to be reached wherever it is stored by an over-the-counter purchaser and can sound or display a simple message instructing the owner to take a specified action. Either terrestrial or extraterrestrial transmission systems can be used and the communication can be point-to-point or by a wide area broadcast.

This application claims the benefit of Provisional U.S. patent application Ser. No. 60/639,476, filed Dec. 27, 2004.

This invention relates to automatic external defibrillators (AEDs) and, in particular, to AEDs which can be sold to individuals over-the-counter (OTC) without a prescription.

Automatic external defibrillators have been in use for a number of years to treat individuals stricken with sudden cardiac arrest, one of the largest causes of death in the United States. Sudden cardiac arrest (SCA) most often occurs without warning, striking people with no previously recognized symptoms of heart disease. It is estimated that more than 1000 people per day are victims of sudden cardiac arrest in the United States alone. SCA results when the electrical component of the heart no longer functions properly causing an abnormal sinus rhythm. One such abnormal sinus rhythm, ventricular fibrillation (VF), is caused by abnormal and very fast electrical activity in the heart. As a result, the heart fails to adequately pump blood through the body. VF may be treated by applying an electric shock to a patient's heart through the use of a defibrillator. The shock clears the heart of abnormal electrical activity (in a process called “defibrillation”) by producing a momentary asystole and providing an opportunity for the heart's natural pacemaker areas to restore normal rhythmic function. When delivered external to the patient, these electrical pulses are high energy pulses, typically in the range of 30 to 360 Joules of energy.

Defibrillators have undergone an evolution over the past decade. Originally defibrillators were manual devices requiring both medical and technical expertise to operate. A physician would carefully set the controls of the defibrillator to apply a shock which diagnosis of the patient or experience with other patients in similar conditions indicated to be most likely to be effective. Following many years of experience with manual defibrillators and motivated by advances in microprocessing and signal analysis, defibrillators have become more automated to the point where a two-pad electrode attached to a patient's chest can detect and diagnose VF and deliver an appropriate shock through the chest wall. However such automated defibrillators continued to be prescription devices used by medical professionals or under the auspices of a controlled emergency response program as described in U.S. Pat. No. 6,694,299. In the final months of 2004 AEDs have reached a level of sophistication and reliability which now enables them to be sold to laypersons without prescription, as over-the-counter (OTC) medical devices. AEDs may now be sold through retail channels (stores, websites, catalogs) and purchased by anyone for use at home in the event of a sudden cardiac arrest emergency.

The use of OTC AEDs poses new challenges when keeping AEDs up-to-date with new and needed features and functions. One of the most critical situations is one in which there is a need to replace potentially faulty software or components. In the past, when AEDs were only sold by prescription from a physician, the manufacturer would receive the name and address of the owner of the AED before it was shipped. If there would be a need to recall the AED for upgrading or repair the manufacturer would have the name and address of the owner on file. But with OTC AEDs, retailers are generally not required to keep purchaser information on file and are often reluctant to establish a system to keep this information or forward it to the AED manufacturer. There is thus a need to be able to contact OTC AED owners when their units need attention or servicing without being able to rely on prescription records commonly used in the past.

U.S. Pat. No. 5,446,678 (Saltzstein et al.) describes a method and apparatus for transmitting an electrocardiogram over an alphanumeric paging network. As the patent points out, paging networks are not designed to transmit large blocks of binary data. Typically only a limited 7-bit character set can be transmitted. To overcome this limitation Saltzstein et al. compress the electrocardiogram data, then break up the compressed data into transmission data blocks which can be accommodated by the paging system. This in turn requires corresponding reconversion and decompression processors at the receiver. The system entails substantial complexity in trying to adapt the limited capability of a paging network to a bandwidth demand for which the network is ill suited.

In accordance with the principles of the present invention the owner of an AED is alerted to the need for servicing or repair by a message which is wirelessly transmitted to and received by the AED. After the message has been received by the AED an alert is issued from the AED which is designed to attract the attention of the owner of the AED. In addition to or as an alternative to issuing an alert, the AED can respond to the message by, for instance, disabling a suspect component or subsystem until needed servicing is performed. The transmitted message can be in the form of one of a limited number of unique codes to which the AED is programmed to respond, thus requiring only a limited bandwidth communication network such as a paging system.

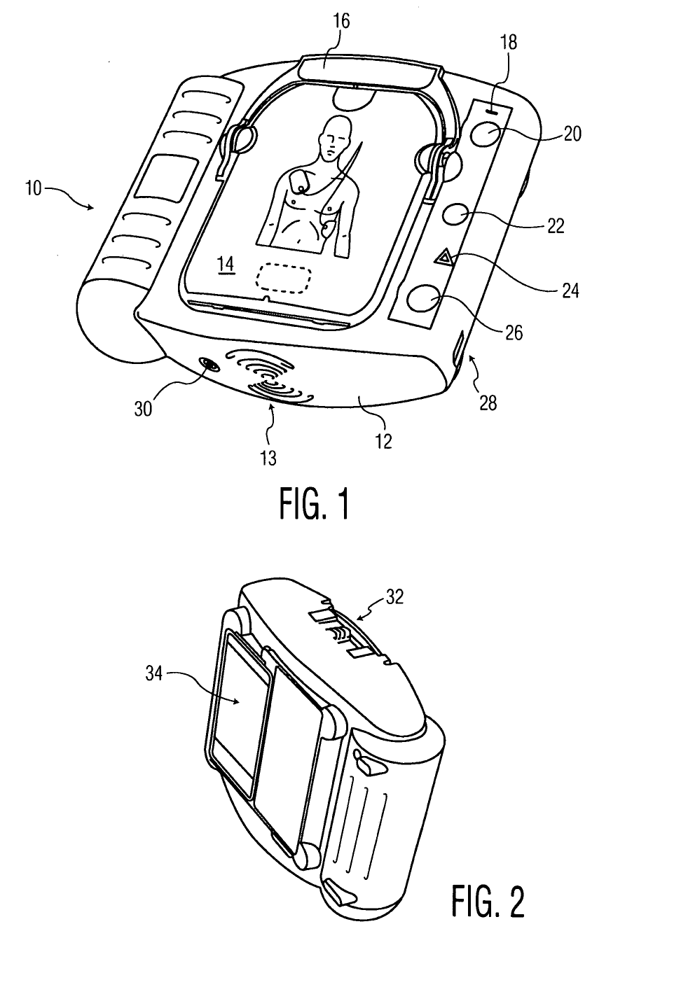

FIG. 1 illustrates a top perspective view of an OTC automatic external defibrillator.

FIG. 2 illustrates a bottom perspective view of the OTC automatic external defibrillator of FIG. 1.

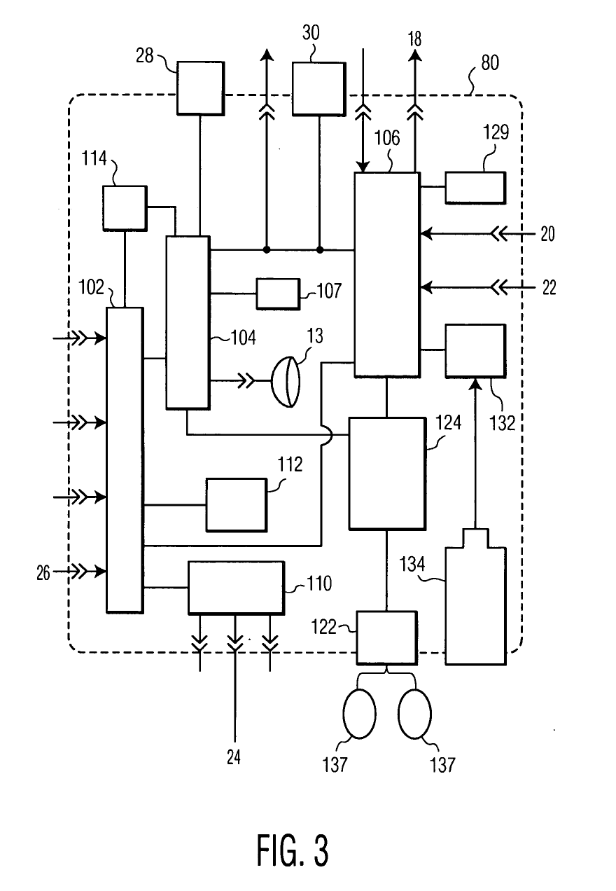

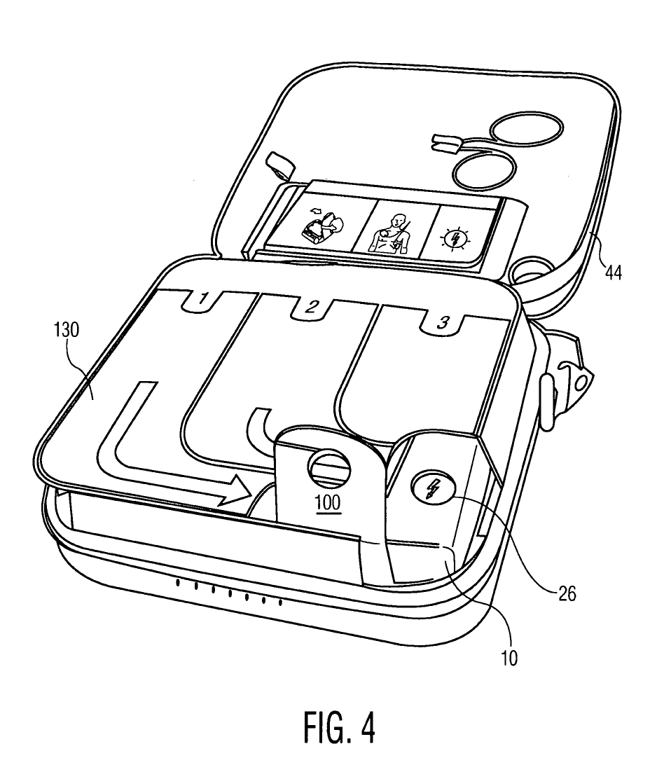

FIG. 3 illustrates in block diagram form the major component parts and subsystems of an AED.

FIG. 4 illustrates an AED in a case which is ready for initial setup.

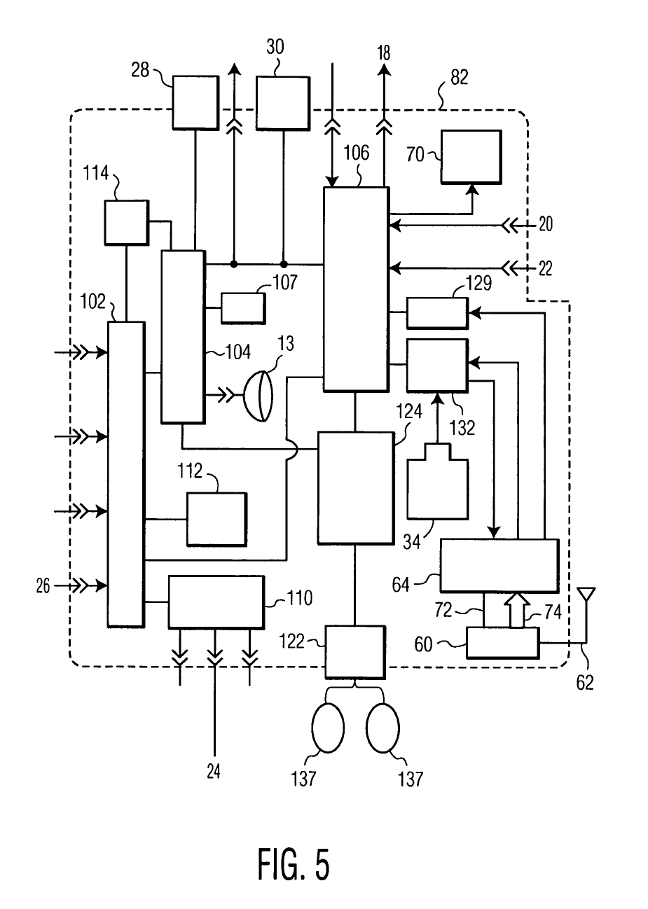

FIG. 5 illustrates an AED constructed in accordance with the principles of the present invention in block diagram form.

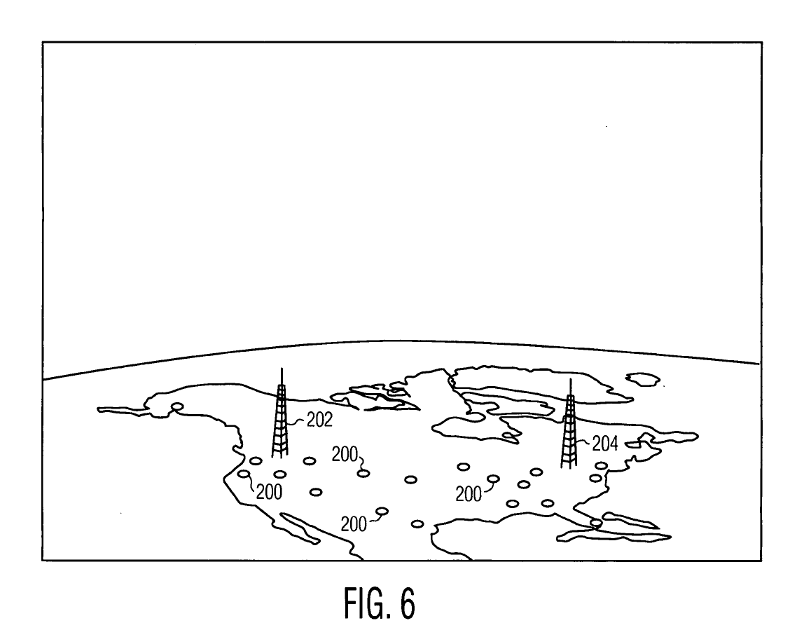

FIG. 6 illustrates a terrestrial communication system for transmitting messages to AEDs over a wide geographic area.

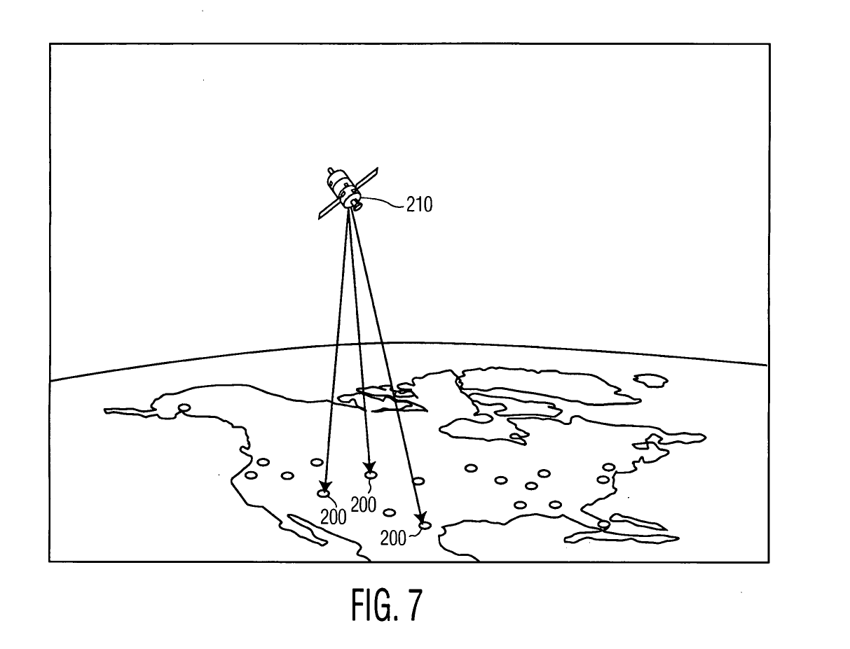

FIG. 7 illustrates an extraterrestrial communication system for transmitting messages to AEDs from a communication satellite.

Referring first to FIG. 1, an OTC AED 10 is shown in a top perspective view. The OTC AED 10 is housed in a rugged polymeric case 12 which protects the electronic circuitry inside the case and also protects the layperson user from shocks. In this embodiment the case is colored a distinctive color which readily identifies the OTC AED to the layperson user, such as red, yellow, orange, green, blue, black, or combinations thereof. Other suitable distinctive colors are light green, silver gray, and various shades of white or off-white. It is important in the home environment that the OTC AED be marked by a prominent color or colors so as to be immediately recognized by a potential rescuer in the event of a home cardiac emergency. Unlike airports and public facilities where AEDs are generally mounted in distinctive locations such as on walls in high traffic areas and with signage to mark and indicate their locations, an OTC AED may be placed anywhere in the home. Since a home OTC AED may go an extended period of time without use, it may be stored in a location lacking prominence such as in a closet or drawer or on a shelf. Accordingly it is very important for the OTC AED to bear a distinctive color as this may be the primary means by which a rescuer can quickly locate the OTC AED in the home during an emergency. The OTC AED may be stored when not in use in a carrying case which may be a distinctive color such as red, black, navy blue, or blue/yellow color.

Attached to the case 12 by electrical leads are a pair of electrode pads. In the embodiment of FIG. 1 the electrode pads are in a sealed airtight cartridge 14 located in a recess on the top side of the OTC AED 10. The electrode pads are accessed for use by pulling up on a handle 16 which allows removal of a plastic cover over the electrode pads. A small ready light, status LED 18, informs the user of the readiness of the OTC AED. In this embodiment the ready light blinks after the OTC AED has been properly set up and is ready for use. The ready light is on constantly when the OTC AED is in use, and the ready light is off when the OTC AED needs attention.

Below the ready light is an on/off button 20. The on/off button is pressed to turn on the OTC AED for use. To turn off the OTC AED a user holds the on/off button down for one second or more. An information button 22 with an “i” on it flashes when information is available for the user. The user depresses the information button to access the available information. A caution light 24 blinks when the OTC AED is acquiring heartbeat (ECG) information from the patient and lights continuously when a shock is advised, alerting the rescuer and others that no one should be touching the patient during these times. Interaction with the patient while the heart signal is being acquired can introduce unwanted artifacts into the detected ECG signal. A shock button 26 is depressed to deliver a shock after the OTC AED informs the rescuer that a shock is advised. An infrared port 28 on the side of the OTC AED is used to transfer data between the OTC AED and a computer. This data port find used after a patient has been rescued and a physician desires to have the OTC AED event data downloaded to his or her computer for detailed analysis.

A speaker 13 provides voice instructions to a rescuer to guide the rescuer through the use of the OTC AED to treat a patient. A beeper 30 is provided which “chirps” when the OTC AED needs attention such as electrode pad replacement or a new battery.

FIG. 2 illustrates another view of the OTC AED 10 in which a cartridge latch 32 is seen on the upper end of the OTC AED. When this latch is pushed to the right the electrode pad cartridge is released from its recess in the OTC AED case 12. The cartridge latch 32 is used when an electrode pad cartridge is to be replaced or exchanged for a training pad set for training on the OTC AED. On the back of the OTC AED case is a battery compartment which houses a battery 34 that powers the OTC AED. In this embodiment the battery 34 is a disposable battery. When the battery 34 becomes discharged, generally after about four years in the readiness state, it is replaced with a fresh battery.

In this embodiment the OTC AED contains self-test circuitry which automatically monitors the state of various parts of the OTC AED on a regular basis. Self-test circuitry is very important for an OTC AED because it cannot be expected that purchasers of the OTC AED will adhere to any formal maintenance schedule for the OTC AED. One component that is self-tested in this embodiment is the battery and another is the electrode pad set. The electrode pads include an adhesive gel which adheres the electrodes to the patient and provides good electrical conductivity with the patient. This adhesive gel is hydrophilic and over time can become subject to desiccation which reduces the effectiveness of the pads. In the hospital setting or the medical emergency responder setting electrode pads are generally used in a relatively short time-frame and desiccation is often not a problem. In addition, these medical professionals are generally more cognizant of the need for attention to expiration dates and other maintenance to their medical equipment. Electrode pads for the prescription defibrillators used by these medical professionals are often not connected to the defibrillator until the defibrillator is to be used and thus cannot be tested by the AED prior to use. Organizations such as airports and office buildings which have deployed defibrillators generally do so under the direction of a medical officer who oversees a maintenance program for the defibrillators. Prescription defibrillators are dispensed under the watchful eye of the prescribing physician who will be mindful of needed periodic maintenance such as electrode pad replacement. In the home environment where the OTC AED is not under the care of a prescribing physician it is to be expected that an OTC AED may sit in readiness for the full two-year anticipated lifetime of a typical electrode pad set without being inspected or used. Accordingly, in one embodiment of the present invention the electrode pads are normally electrically connected to the OTC electronic unit 10 and its self-test circuitry while the OTC AED is in the readiness state. With an electrode pad cartridge this can be done by embedding conductors in the wall of the cartridge. The electrode pad leads inside the cartridge are connected to these conductors, which enables electrical connectivity to the exterior of an air-tight sealed cartridge. The cartridge conductors engage mating conductors in the recess of the OTC AED case, thereby putting the electrode pads into electrical communication with the OTC AED self-test circuitry. In other embodiments the pad may be sealed in an air-tight wrapping with the electrode leads and connector extending from the wrapping. The pad connector is connected to a mating connector on the AED unit 10. These connections permit the electrode pads to be automatically tested by the OTC AED on a periodic basis by measuring the impedance through the circuit which includes electrical leads to each electrode pad, the conductor of each electrode, and the conductive gel on each electrode conductor. If the self-testing determines that the electrode pads have dried out or suffered some other detected deterioration as by an impedance measurement which is outside an expected impedance range, the user is alerted to replace the pads by the chirping of the beeper 30 and the absence of the ready light 18. Further details of electrode self-testing may be found in U.S. Pat. No. 5,879,374, to Powers, et al. for “External Defibrillator with Automated Self-Testing Prior to Use” and U.S. Pat. No. 6,694,193, the specifications of which are incorporated herein by reference.

FIG. 3 is a schematic of the various components, subsystems, and interconnections of a typical AED 80. In this illustration, defibrillator control functions are divided among a microprocessor unit (MPU) 102, an application-specific integrated circuit (ASIC) 104 and a system monitor 106. MPU 102 performs program steps according to software instructions provided to it from memory 114 which may comprise one or more of EPROM, RAM and flash ROM memory. MPU 102 controls the operation of certain system LEDs through an LED control circuit 110, including an LED associated with the shock button 26, the LED associated with the Do Not Touch indicator 24, and LEDs which indicate the body locations where the electrode pads are to be placed, on units so equipped. MPU 102 also receives system status information as shown by block 112 which is sent to status circuit 129, temperature information from the interior of the case 12 from a temperature sensor (not shown), and a signal from a sensor when training pads are plugged into the pad connector 122. The training pad sensor can be a magnetic sensor associated with connector 122 which senses the field of a small magnet integrated into the connector of a training electrode pad set, for example. The MPU is also responsive to a signal from a pedi-key sensor associated with a slot into which a pedi-key is inserted to switch the operation of the AED unit to a pediatric rescue protocol, as described in U.S. patent appl. No. 60/637,682.

ASIC 104 implements a memory map to system memory 114. ASIC 104 is clocked by a clock 107 and also controls the speaker 13 which delivers audible instructions during use of the AED. ASIC 104 can actuate a relay within the shock delivery and ECG front-end system 124 in response to actuation of the shock button 26 by a user during treatment. ASIC 104 will actuate an LED associated with the information button to signal to the user that information is available and can be accessed by depressing the information button 16. The ASIC also provides the interface to the IR port 28 through which new program information can be loaded into the AED unit and rescue data can be communicated to another data storage or analysis system.

System monitor 106 performs automatic self-tests of the AED and its components as described previously. The system monitor 106 controls the status LED 18 to indicate that the self-tests are showing proper system operation, and activates beeper 30 to provide an audible alert when the system is not operating properly. System monitor 106 is also the defibrillator's interface with the on/off button 20, the information button 22, and a sensor associated with pad connector 122 which signals the connection of a specific type of electrode pad 137 to the AED unit. System monitor 106 controls a power management subsystem 132 to provide power to operate system components from power supply 34 and to provide energy to the shock delivery system's capacitor(s) for a therapeutic shock during treatment. System monitor 106 also interfaces with the defibrillator's ECG front end, enables the shock delivery system to deliver a shock in response to detection of a patient ECG pattern requiring treatment (and actuation of the shock button 26), and controls delivery of the shock to electrode pad connector 122 in response to shock delivery status information (e.g., patient impedance) obtained during delivery of the shock. Further information regarding this last function may be found in U.S. Pat. No. 5,735,879 to Gliner et al. for “Electrotherapy Method for External Defibrillators,” and U.S. Pat. No. 5,607,454, to Cameron et al. for “Electrotherapy Method and Apparatus,” the specifications of which are incorporated herein.

As described previously, electrode pad connector 122 may communicate directly with the system monitor 106 to identify the electrode type, or connector 122 may communicate with system monitor 106 via an identifier receiver that interfaces between the system monitor and the identifier of the electrode pad connector 122. For example, in an optical encoding embodiment, photodetectors could act as an identifier receiver in communication between the system monitor and the electrode pad connector 122.

These defibrillator components communicate with each other over suitable communication buses, as shown.

When defibrillators are shipped to purchasers the units are shipped without the battery being installed. The units are not shipped with the batteries installed because of the possibility of inadvertent activation and the resultant hazard if the high voltage circuitry begins the charge the defibrillator capacitor to its usual level of hundreds or thousands of volts. It is also possible that a self-test performed during shipment could detect an error condition, causing the defibrillator to issue its audible alerts for maintenance during shipment, a situation to be avoided for obvious reasons such as airline safety. After the defibrillator is received by the purchaser, the first action of the medical professional is to install the battery in the defibrillator, at which point the defibrillator usually performs a self-test known as a “battery insertion test.” This process begins the setup of the defibrillator, which may require periodic intervention by the medical professional before setup is complete. As mentioned above, it is important that the OTC AED be promptly set up when the layperson purchaser takes it home. Furthermore, it is desirable to make setup as simple as possible for the nonmedical layperson. FIG. 4 illustrates an AED for which initial setup is simplified by providing the AED with its battery already installed, alleviating the layperson of this task. However, to prevent inadvertent charging of the high voltage circuitry and capacitor during shipment, the battery circuit is broken by a nonconductive pull tab 100 during shipment. The distal end of the pull tab 100 is disposed in the battery circuit such as between one battery terminal and its contact on the AED. In a constructed embodiment the battery has four terminals which engage four contacts on the AED, and the distal end of the pull tab 100 is disposed between all four terminals and contacts, completely isolating the battery from the high voltage circuitry of the AED. The pull tab 100 may be made of a sheet of nonconductive material such as paper or cardboard. In a constructed embodiment the pull tab 100 is made of a thin polymeric sheet which is tough enough not to tear when a finger is inserted in the hole in the proximal end of the pull tab and the pull tab is pulled from between the battery terminals and AED contacts. The thin sheet enables the battery to be latched in place in the battery compartment while the pull tab is in place during shipment. The polymeric material also gives the pull tab a resilient property. During shipment in a constructed embodiment the pull tab is folded over the top of the OTC AED when the OTC AED is in the carrying case 44, and the case is closed. When the case is opened for the first time the resilient pull tab 100 pops up, immediately informing the layperson what is to be done first. The pull tab may be labeled with instructions at its proximal end such as “pull” or “remove first”, or it may be labeled with a graphic such as an arrow pointing up, or it may be left unlabeled with the pop-up characteristic speaking for itself.

While the installed battery is a benefit because it alleviates the layperson purchaser of this task, it is also an advantage because the OTC AED packaging does not have to accommodate a separate battery pack and thus can be made smaller. In a constructed embodiment the AED with battery installed and in the carrying case 44 measured 3½ ″ by 8″ by 9″, a total of 252 in³, and was packed in packaging measuring 6.5″ by 8.375″ by 10.5″, a total of 572 in³. The OTC AED and case thus occupy 44% of the packaging volume.

In the illustrated embodiment of FIG. 4 the top of the AED is covered with a sheet 130 that obscures from the user certain ones of the controls of the AED, seen in FIG. 1, except for those that are to be used to initially set up the AED. The sheet 130 covers the top of the AED and contains three instructions: pull the red tab 100 to start the automated setup process; press the orange button 26 indicated by the second arrow when prompted by an audible instruction; and wait until an audible prompt announces that the setup is complete. When the new purchaser opens the carrying case 44 for the first time, the pull tab 100 pops up and the purchaser responds by pulling the tab, connecting the battery terminals to the contacts of the OTC AED. The OTC AED will then immediately commence its battery insertion test, and the audible prompts may announce to the purchaser that testing is underway. At the conclusion of the battery insertion test, during which no user intervention is needed, the purchaser is asked to respond by pressing the shock button 26. At this point no other controls are visible to the purchaser by reason of the cover sheet covering the top of the OTC AED controls except for the shock button. The cover sheet 130 also inhibits the purchaser from pressing any other buttons on the OTC AED while the battery insertion test is in progress. In the illustrated embodiment the cover sheet also obscures the pull handle for the pads cartridge as it is not necessary for the purchaser to pull this handle during setup. When the battery insertion test is complete the purchaser can dispose of the pull tab 100 and the cover sheet 130 and the OTC AED is set up and ready for use in a cardiac emergency.

FIG. 5 illustrates an AED 82 with a wireless receiver 60 constructed in accordance with the principles of the present invention. In one embodiment the receiver 60 is a low bandwidth receiver for receiving short messages of simple commands or codes. One simple command or code for instance can be to put the AED 82 into an alert status, whereby the owner is alerted that servicing is needed. Another simple command or code can be to inhibit operation until servicing is performed, for example. To simplify the receiver 60 even further, the receiver 60 does not need to have a transmit mode; it only needs to be able to receive information. In other embodiments the receiver 60 may be a transceiver which, for instance, acknowledges the receipt of a message or polls the wireless communications system for pending messages. In other embodiments the receiver 60 may have a broad bandwidth capable of receiving long or complex messages such as data transmissions of software which remedy a detected problem.

The receiver 60 has an antenna 62 for reception of wireless messages. The receiver is coupled to a stand-by processor or ASIC 64 which is continually powered by power management subsystem 132 at a low level to detect the receipt of a message by the receiver. The receiver 60 is coupled to the stand-by processor/ASIC 64 by control lines 72. When the receiver is of the form of a modem or telephone receiver the control lines 72 provide a means by which the AED will answer an incoming call, for instance. The control lines 72 also can provide a means for the receiver to alert the stand-by processor/ASIC 64 that a message has been received. In this embodiment a received message is coupled to the stand-by processor/ASIC 64 over data lines 74. In another embodiment the data and control signals may be multiplexed over a common line or lines.

In an alternate embodiment, the receiver 60 is a transceiver which is unpowered until activated by system monitor 106. In this embodiment, system monitor 106 periodically activates receiver 60 via stand-by processor/ASIC 64, such that receiver 60 identifies itself to the communication system, and polls the communication system for pending messages. The communication system responsively transmits pending messages to receiver 60. If no messages are pending, receiver 60 is again inactivated.

In the above embodiments, the received message is decoded by the stand-by processor/ASIC 64 which identifies the action to be taken by the AED. Generally the message will require the activation of other subsystems of the AED 82 and the stand-by processor/ASIC 64 is coupled to the power management subsystem to command it to power up other subsystems such as the system monitor 106 and the status circuit 129. The decoded message is then sent to the status circuit 129. Alternatively in another embodiment the received message can be coupled to the status circuit for decoding and the status circuit will then command the appropriate circuits to be powered up for response.

The status circuit 129 is coupled to the system monitor 106 to cause the AED to take the necessary action. For instance, if the ready light 18 normally blinks when the AED is ready for use, the system monitor 106 may turn the ready light off to indicate that the AED needs attention. In other embodiments where the AED has a display panel the visual alert can be displayed on the display panel. In still other embodiments the color of the ready light can be changed from green to orange or some other warning color, for example. The system monitor may also cause the ASIC 104 to issue an audible alert through the speaker 13 such as “ATTENTION REQUIRED!” or by actuating the beeper 30. An audible alert may be necessary in the instance where the AED is stored out of a user's sight such as in a closet or drawer where the ready light is not normally visible. The audible alert can be repeated periodically until the owner responds by providing the required attention to the AED.

In this embodiment the AED 82 has an LCD display 70 which is coupled to the system monitor for the display of information prompted by the receipt of messages by the receiver 60. The LCD display can be an inexpensive one-line display for the display of simple messages. For instance the LCD display can display the message “PRESS INFORMATION BUTTON i”. When the owner then presses the information button 22 an audible message is played through the speaker 13, informing the owner of the action needed. As another example, the LCD display 70 can display a message such as “CALL 1-800-[toll-free number]”, thereby instructing the owner to call a toll-free service telephone number where the owner may receive detailed instruction as to the action needed.

Any suitable wireless system and compatible receiver can be used for the AED 82. In embodiments in which only short codes or commands are used, pager technology is highly suitable. SMS paging technology is reliable, well-developed, inexpensive and has low power requirements. An AED is sold with a unique pager telephone number which is linked to the serial number of the unit, enabling the manufacturer or service person to uniquely contact a specific AED or group of AEDs. Wireless cellular telephony may be used, such as GSM, TDMA or CDMA which also enables point-to-point communication with a specific AED or group of AEDs. Another wireless technology which may be employed in a constructed embodiment is wireless Internet technology. In such an embodiment an AED can have a unique IP address with messages being sent to the AED over a wireless data network. In other embodiments wide area broadcast technology may be employed. One such technology which may be used is “In-Band, On-Channel” (IBOC) digital broadcasting technology by which data is broadcast over AM or FM bands. Such technology may be desirable because it uses the existing radio broadcast infrastructure and is capable of reaching a broad coverage area. Low power, low cost IBOC receivers are available from companies such as Texas Instruments, Inc. and Lucent Technologies.

While wireless networks are preferred for home use, since they require no wiring or installation by the home user, it will be appreciated that in certain embodiments wired communication may be desirable. For instance, an AED may be mounted on a wall bracket or other fixture which has a telephone or data line associated with it. The AED may thereby be connected to a land line telephone system or data network, receiving messages over the lines connected to the AED's storage location.

An example of such a terrestrial AED communication system is shown in FIG. 6. A terrestrial transmitter 202 transmits wireless signals which are received by one or more AEDs at locations 200. As the drawing illustrates, multiple transmit locations 202, 204 can be used to reach AEDs disbursed over a wide geographic area.

Extraterrestrial communication such as a satellite-based broadcast system may also be employed in a given embodiment as illustrated by FIG. 7. As this drawing illustrates, messages are transmitted to AEDs at locations 200 by a satellite transmitter 210. An extraterrestrial system such as this can be used to reach AEDs distributed over a wide geographic area.

In addition to being used for service messages as described above, a system of the present invention can be used for response messages. For instance, the owner of an AED living in a neighborhood can be called to respond to cardiac emergencies at other homes in the neighborhood. A message can be sent to the owner's AED from an emergency response center for instance, causing the AED to issue an audible response alarm, and the address where the AED is needed can be displayed on the LCD display 70. As another example, an AED may be stored on a wall bracket in the security office of an office building where the security personnel are trained responders. The phone line or data line connected to the AED could deliver a message such as “FLOOR 8, ROOM 812” on the LCD display, notifying the trained responders where the AED is needed.

Another type of message which may be used is a maintenance message. As previously mentioned it is to be anticipated that the consumer purchaser of an OTC AED will be unmindful of any maintenance needs of the OTC AED while it is in its standby state. A message can be sent to the AED periodically requesting that the condition of the electrode pads be tested, or the charge of the battery be checked. Such messages can augment the self-testing of these components normally carried out by the AED.

Other types of messages which can be sent are reminder or locating messages. For example, the owner of an OTC AED may wish to be reminded annually of the location of his AED, which can be useful when the AED is stored out of sight. A message can be sent to the AED annually, causing the AED to issue an audible alert which causes the owner to find his AED. The owner is thus reminded of the location where he has stored the AED. Such messages can also be used to locate the AED when the owner forgets where the AED is stored. The owner can call the manufacturer or a service center and ask that an alert be sent to his AED. The message will be sent and the AED will issue its audible alert, enabling the owner of the OTC AED to locate the device in the home or other location.

1. An automatic external defibrillator (AED) which is stored at a location for emergency use in the future comprising:
a power supply;
a high voltage circuit coupled to the power supply;
a user alert device;
a processor operable to issue user alerts of the need for servicing the defibrillator by means of the user alert device; and
a receiver, coupled to the processor, and responsive to a message received from an external source indicating the need for servicing of the defibrillator,
wherein the user alert device issues an alert indicating the need for servicing of the AED.

2. The automatic external defibrillator of claim 1, wherein the user alert device comprises a beeper.

3. The automatic external defibrillator of claim 1, wherein the user alert device comprises a speaker which issues voice messages.

4. The automatic external defibrillator of claim 1, wherein the user alert device comprises a visual display.

5. The automatic external defibrillator of claim 4, wherein the visual display comprises an LCD display.

6. The automatic external defibrillator of claim 1, wherein the receiver comprises a wireless receiver.

7. The automatic external defibrillator of claim 6, wherein the wireless receiver comprises at least one of a paging message receiver, a cellular telephone receiver and a Internet message receiver.

8. The automatic external defibrillator of claim 1, wherein the receiver comprises a non-wireless receiver.

9. The automatic external defibrillator of claim 8, wherein the non-wireless receiver comprises at least one of a telephone signal receiver and an Internet signal receiver.

10. The automatic external defibrillator of claim 1, wherein the receiver further comprises a coded message receiver;
wherein the processor comprises a coded message decoder,
wherein the user alert device issues an alert corresponding to the decoded message.

11. The automatic external defibrillator of claim 1, wherein the automatic external defibrillator comprises an AED which is sold to a user without a prescription.

12. The automatic external defibrillator of claim 11, wherein the AED is stored at a residence.

13. (canceled)

14. An automatic external defibrillator (AED) which is stored at a location for emergency use in the future comprising:
a power supply;
a high voltage circuit coupled to the power supply;
a user alert device;
a processor operable to issue user alerts of the need for maintenance of the AED by means of the user alert device; and
a receiver, coupled to the processor, and responsive to a message received from an external source indicating the need for maintenance of the AED,
wherein the user alert device issues an alert indicating the need for maintenance of the AED.

15. (canceled)

16. The automatic external defibrillator of claim 1, wherein the receiver is further responsive to a message indicating the need to identify the location of the AED,
wherein the user alert device issues an alert indicating the location of the AED.

17. A method for alerting an individual to the need for servicing and AED which has a message receiver comprising:
transmitting a message for reception by the AED message receiver;
receiving the message by the AED message receiver;
identifying in the AED a servicing action to be taken by a user in response to the message; and
issuing an alert to a user by the AED of the action to be taken.

18. The method of claim 17, wherein issuing further comprises issuing at least one of a visual and an audible alert by the AED.

19. The method of claim 18, further comprising changing the status of the AED in response to the message.

20. The method of claim 17, wherein transmitting comprises transmitting a message by a terrestrial transmission system.

21. The method of claim 20, wherein the terrestrial transmission system comprises at least one of a paging system, a telephone system, an Internet system and a radio system.

22. The method of claim 17, wherein transmitting comprises transmitting a message by an extraterrestrial transmission system.

23. A method for an AED with a message receiver to receive messages pertaining to possible service needs comprising:
powering up the message receiver;
sending a message with the receiver which identifies the AED to a communication system;
polling the communication system for pending messages for the AED;
receiving a message if one is pending;
powering down the message receiver; and
taking an action in response to the received message.

24. The method of claim 23, wherein taking an action further comprises decoding the received message by the AED to determine the action to be taken.

25. The method of claim 23, wherein the steps of powering up through powering down are performed periodically by the AED.

26. The method of claim 23, wherein taking an action includes notifying an individual that the AED needs attention.
